(19) United States
(12) Patent Application Publication
(10) Pub. No.: US 2015/0320836 A1
(43) Pub. Date: Nov. 12, 2015

(54) CRYOPRESERVATION OF CELLS INSIDE A MACRO-ENCAPSULATION DEVICE

(71) Applicant: The Regents of the University of California, Oakland, CA (US)

(72) Inventors: Pamel Itkin-Ansari, Carlsbad, CA (US); Igor Katkov, La Jolla, CA (US)

(21) Appl. No.: 14/410,557

(22) PCT Filed: Jul. 3, 2013
(86) PCT No.: PCT/US2013/049381

§ 371 Date: Dec. 22, 2014

Related U.S. Application Data
(60) Provisional application No. 61/668,982, filed on Jul. 6, 2012.

(51) Int. Cl. A61K 38/28; A61K 35/545; A61K 35/39

(52) U.S. Cl. CPC A61K 38/28; A61K 35/39; A61K 35/545

(57) ABSTRACT

Embodiments disclosed herein relate to methods and compositions to simplify creation, storage, distribution, and Encapsulation Technology use of encapsulated cell based therapeutics, particularly cell-based therapeutics comprising islet-like cell clusters.

### RELATED APPLICATION

The present application claims priority to U.S. Provisional Application Ser. No. 61/668,982, filed Jul. 6, 2012, which is hereby incorporated by reference in its entirety.

### BACKGROUND OF THE INVENTION

Currently, few diabetic patients receive islet transplantation due to a shortage of islet cells and the need for lifelong immunosuppression following transplantation. The present inventors have demonstrated that a macro-encapsulation device protects murine islets from immune rejection. They have also determined that encapsulated human ES derived pancreatic epithelium matures in vivo and allows for the treatment of diabetes in animals. In addition, the inventors have demonstrated that subcutaneous placement of cell-filled devices makes this therapy minimally invasive.

Current islet transplantation protocols require patients to travel to a limited number of medical centers where cells are administered into the hepatic portal system. A major problem with current transplantation protocols is the inability to deliver cells immediately or for them to be kept fresh for a few days prior to transplantation.

Patents covering material related to the present disclosure include U.S. Pat. No. 5,344,454, relating to closed porous chambers for implanting tissue in a host; U.S. Pat. No. 5,593,440 relating to Tissue implant systems and methods for sustaining viable high cell densities within a host; U.S. Pat. No. 5,453,278 relating to Laminated barriers for tissue implants; U.S. Pat. No. 4,816,339 relating to Multi-layered poly(tetrafluoroethylene)/elastomer materials useful for in vivo implantation; U.S. Pat. No. 5,713,888 relating to Tissue implant systems; and U.S. Pat. No. 5,964,261, Implantation Assembly, which claims the use a 2nd bag to encase a first bag for storage, culturing, transportation or cryopreservation. Also relevant are more recent patents U.S. Pat. No. 7,820,195, U.S. Pat. No. 7,361,333, and U.S. Pat. No. 6,638,765.

Alginate micro-encapsulation is one of the most widely accepted methods and cryopreservation of micro-encapsulated cells has been achieved including stem cells and neurospheres (Malpique et al. (2010) Tissue Eng Part C Methods, 16:965-977; Sambu et al. (2011) Proc Inst Mech Eng H, 225:1092-107; Serra et al. (2011) PLoS One, 6:e23212). The authors also built a mathematical model of the survival rate of mouse embryonic stem cells (Sambu et al. (2011) Proc Inst Mech Eng H, 225:1092-107). The majority of islet encapsulation studies to date have been performed with microcapsules which contain one or a few islets, thereby providing a beneficial surface/volume ratio for diffusion (Beck et al. (2007) Tissue Eng, 13:589-599; von Mach et al. (2003) Acta Diabetol, 40:123-9). Cryopreservation of rat pancreatic islets micro-encapsulated in the alginate matrix has been explored by Schneider and Klein. They used an elaborative protocol that included stepwise addition of the cryoprotective agent (dimethyl sulfoxide, DMSO; final concentration 2.0M), and a stepwise removal of DMSO by sucrose dilution after thawing, as well as controlled programmable freezer similar to the protocol that was used by von Mach for non-encapsulated rat islets (von Mach et al. (2003) Acta Diabetol, 40:123-9). High recovery rates of cryopreserved islets have been achieved with long-term insulin secretion capacity and graft function (up to 52 week). (Zimmermann et al. (2007) Curr Diab Rep, 7:314-320; Zimmermann et al. (2005) J Mater Sci Mater Med, 491-501).

### SUMMARY OF INVENTION

Embodiments disclosed herein relate to methods to simplify creation, storage, distribution, and use of encapsulated cell based therapeutics.

In some embodiments, a method of cryopreserving viable cells comprising contacting said cells with DMSO and freezing the islet like cell clusters is disclosed. In some embodiments the cells secrete a therapeutic compound, such as a protein or small molecule therapeutic compound. In some embodiments the cells comprise insulin expressing/pancreatic epithelial cells. In some embodiments the cells comprise secretory protein expressing cells, such as cells expressing colony-stimulating factors, erythropoietin, growth hormone, insulin, interferon, human growth factor (“HGF”), or plasminogen activators. In some embodiments the cells comprise stem cells, such as human embryonic stem cells (hESCs). In some embodiments the cells are islet like cell clusters (“ICCs”). In some embodiments the methods comprise contacting said islet like cell clusters with DMSO and freezing the islet like cell clusters is disclosed. One aspect of these embodiments involves thawing the islet like cell clusters such that said thawed ICCs comprise viable cells. One aspect of these embodiments involves DMSO as a constituent of a composition that comprises 10% DMSO. One aspect of these embodiments involves transporting the frozen islet like cell clusters to a clinical setting. One aspect of these embodiments involves islet like cell clusters that are preloaded into a cryopreservation device before freezing. One aspect of these embodiments involves a cryopreservation device that is a macroencapsulation device. One aspect of these embodiments involves cells that are frozen before loading into a cryopreservation device. One aspect of these embodiments involves freezing that is partial. One aspect of these embodiments involves freezing that is total. One aspect of these embodiments involves transplanting the islet like cell clusters into a patient. One aspect of these embodiments involves transplantation that is subcutaneous. One aspect of these embodiments involves cells that are maintained in DMSO for up to 20 minutes after thawing. One aspect of these embodiments involves cells that are maintained at a temperature of up to 37° C. for up to 20 minutes.

In some embodiments, a composition involves DMSO and islet like cell clusters. One aspect of these embodiments involves a composition that is frozen and islet like cell clusters that are viable. One aspect of these embodiments involves a cryopreservation device. One aspect of these embodiments involves a cryopreservation device is impermeable to the islet like cell clusters. One aspect of these embodiments involves cryopreservation device is permeable to at least one substance secreted by the islet like cell clusters. One aspect of these embodiments involves insulin being secreted. One aspect of these embodiments involves a cryopreservation device that is configured to maintain its integrity upon subcutaneous introduction into a mammal.

In some embodiments, a macro encapsulation device involves frozen islet like cell clusters. One aspect of these embodiments involves at least about 30% of the cells in said islet like cell clusters being viable after thawing.

### BRIEF DESCRIPTION OF THE DRAWINGS

FIG. 1A. DMSO Toxicity in Monolayers of Insulin Expressing Cells without Freezing. Legends: 1—NO DMSO; 2—Washed immediately after contact with 4° C. 10% DMSO; 3—DMSO incubation for 10 min at 4° C.; 4—30 min×4° C.; 5—10 min×37° C.; 6—30 min×37° C. Treatments differ significantly if they do not share a common letter (p<0.05).

FIG. 1B. Freezing Insulin-Expressing Cells in Monolayers: Effects of Pre-Freeze Incubation with DMSO. Legends: 2—frozen without DMSO; 3—DMSO incubation for 10 min at 4° C.; 4—30 min×4° C.; 5—10 min×37° C.; 6—30 min×37° C. Treatments differ significantly if they do not share a common letter (p<0.05).

FIG. 2. Freezing ICCs: Effects of Pre-Freeze DMSO Toxicity and Centrifugation (N=3). 1—Control (37° C.×20 min., CFG w/o freezing); 2—NEG. Control (Frozen, no DMSO, NO CFG before freezing); 3—25° C.×20 min., NO CFG CFG before freezing; 4—25° C.×20 min, CFG before freezing; 5—37° C.×20 min, NO CFG before freezing; 6—37° C.×20 min, CFG before freezing. CFG=centrifugation. Treatments differ significantly if they do not share a common letter (p<0.05).

FIG. 3A. Post-Thaw DMSO Toxicity after Freezing ICCs in Cryovials (N=6 Legends: 1—Control (Nonfrozen, No DMSO); 2—THAWED and diluted immediately; 3—THAWED 25° C.×10 min.; 4—THAWED, 25° C.×20 min.; 5—THAWED, 4° C.×20 min.; 6—THAWED, 37° C.×20 min. Treatments differ significantly if they do not share a common letter (p<0.05).

FIG. 3B. Insulin Expressing after Freezing ICCs in Cryovials (N=6). Insulin-GFP was evaluated in control cells (Left), and in cells exposed to 10% DMSO for 20 min 37° C. imaged for GFP alone (Middle) or for GFP and DAPI (Right).

FIG. 4A. Cryopreservation in an immuneisolated macro-encapsulation device—schematic view and working principle.

FIG. 4B: Microscopic view of mesh network and encapsulated cells (adapted with permission).

FIG. 4C: TheraCyte™ device suspended in a wash buffer for continuous laminar flow elution of DMSO after freezing with a magnetic stirrer at the bottom.

FIG. 5. ICCs Frozen in TheraCyte™ Encapsulation Device (N=2). Legends: 1—Control (Nonfrozen cells in device); 2—Cells frozen inside device. Treatments differ significantly if they do not share a common letter (p<0.05).

FIG. 6. Overall Comparison of Different Experiments. Summary of best selected treatments from all Experiments 1-4 based on the Viable Yield Yᵥ (f-la). M-N: Monolayer (dissociated), non-frozen; M-F: Monolayer (dissociated), frozen in a cryovial; C-N: Clusters, non-frozen; C-F: Clusters, frozen in a cryovial; CN-D: Clusters, non-frozen, loaded in the DEVICE; C-F-D: clusters, frozen in the DEVICE. Treatments differ significantly if they do not share a common letter (p<0.05).

### DETAILED DESCRIPTION

To begin addressing the issues involved in cryopreservation of macro-encapsualted cells, Ins-G cells were employed, a cell line derived from human fetal islets (Kiselyuk et al. (2010) J Biomol Screen, 15:663-670). Importantly, islets were not chosen for these studies as they are not actually relevant to an ultimate goal which is to encapsulate robust progenitor cells and may be extremely susceptible to these manipulations. In some embodiments, if progenitors are transplanted, the islets which mature inside the device may never be subjected to DMSO exposure.

The present embodiments relate to methods for cryopreservation of cells preloaded into an encapsulation device. The present embodiments allow for a plurality of cells, in encapsulation devices, to be saved for future use. In some embodiments the cells are insulin expressing/pancreatic epithelial cells. In addition, the present embodiments also provide a means, method or device for shipping cells and a means, method or device for simplifying procedures at the transplantation center. As an alternative for the transplantation center to fill devices with cells, the cells are pre-packaged and ready to be thawed and transplanted (or possibly transplanted in the frozen state). Thus, entire therapeutic units, consisting of encapsulated cells could be shipped to physicians. The simplicity should allow many physicians to treat patients instead of patients having to travel to a limited number of transplant centers.

Many clinical conditions, deficiencies, and disease states can be remedied or alleviated by supplying to the patient one or more biologically active molecules produced by living cells or by removing from the patient deleterious factors which are metabolized by living cells. In many cases, these molecules can restore or compensate for the impairment or loss of organ, cellular or tissue function. Accordingly, many investigators have attempted to reconstitute organ, cellular or tissue function by transplanting whole organs, organ tissue, and/or cells, which provide secreted products or affect metabolic functions. However, while transplantation can provide dramatic benefits, it is limited in its application by the relatively small number of organs that are suitable and available for grafting. In general, transplantation patients must be immunosuppressed in order to avert immunological rejection of the transplant, which results in loss of transplant function and eventual necrosis of the transplanted tissue or cells. Likewise, in many cases, the transplant must remain functional for a long period of time, even for the remainder of the patient's lifetime. It is both undesirable and expensive to maintain a patient in an immunosuppressed state for a substantial period of time. Embodiments disclosed herein been shown to provide both allograft protection and autoimmune protection in rodents and allograft protection in primates.

Some embodiments relate to a method for freezing cells inside a macroencapsulation (>500 cells) device. In particular, some embodiments relate to a method for cryopreservation of insulin expressing/pancreatic epithelial cells preloaded into an encapsulation device. Thus far, we have encapsulated a cell line derived from human islets (T6PNEinsGFP) and frozen the combined cell/device. We propose that this method will be useful, for example, for encapsulated human ES cells derived pancreatic epithelium for the treatment of diabetes in humans without immunosuppression. In addition, the subcutaneous placement of cell-filled devices makes this therapy minimally invasive. Cells can be preloaded prior to freezing of the device and/or cells can be partially or fully frozen before loading.

In methods for cryopreservation of cells inside a macro-encapsulation immunoisolating device, an entire therapeutic unit (cells in device) can be stored and transported to clinical settings for transplantation. In some embodiments, the device is a TheraCyte™ encapsulation device. In some embodiments, the device is available from TheraCyte, Inc. (Laguna Hills, Calif.). This method will minimize the manipulation necessary for clinicians to transplant the cells. The embodiments disclosed herein allow for quality control and should increase patient access to the therapy. Storing frozen cells within a device will provide quality control as all devices will be filled and stored in specialized banks as a measure of quality control. Second, embodiments disclosed herein allow for a method for simple distribution and storage of therapeutic agents. Because shipped devices will be preloaded with cells, the clinician/transplant center will not have to load devices. This will allow for simple application to the patient. Macro-encapsulation units can be shipped and stored frozen until use.

Some embodiments also allow the encapsulated cells to be transplanted for diagnostic, therapeutic purposes or transplantation of encapsulated insulin producing cells (or their progenitors) in preclinical studies. Potential therapies for transplanted cells include but are not limited to stem cell transplantation, diabetes therapy and/or transplantation of any cells making a secreted factor. Additionally, an encapsulation device provides a vessel for containment of transplanted cells in vivo. The device may or may not be retrievable and may or may not be immunoisolating. Freezing techniques may be replaced by vitrification/dessication or any method for preservation by inducing a low or non-metabolic state in encapsulated cells.

Some embodiments propose that freezing cells in devices will provide superior quality assurance, storage, disbursement, and potentially, use of encapsulated cell based products. The present disclosure would improve patient care, as physicians would not need access to cultured cells and/or GMP facilities for loading devices. Instead, physicians would be able to transplant a partially or fully prepared therapeutic unit.

An encapsulated cell based therapy for the generation of a secreted protein is attractive. In some embodiments the therapy presents a cell population generative of the secreted protein of interest. In some embodiments the therapy separates a cell population from the recipient's immune system such that the cell based therapy may be maintained in the body of the recipient without administration of an ongoing immunosuppressant regime. In some embodiments the therapy separates potentially oncogenic cell populations from the therapy recipient such that the risk of the deleterious establishment or spread of the potentially oncogenic cell population is minimized.

An encapsulated cell based therapy for diabetes is especially attractive due to the cell autonomous nature of pancreatic islets and the fact that only a single, small, secreted product (insulin) is required (Schneider et al. (2005) Diabetes, 54:687-691).

An encapsulated cell based therapy is also especially attractive if the cells comprise secretory protein expressing cells, such as cells expressing colony-stimulating factors, erythropoietin, growth hormone, insulin, interferon, HGF, or plasminogen activators. Accordingly, in some embodiments the cells comprise stem cells, such as human embryonic stem cells (hESCs). In some embodiments the cells are islet like cell clusters (“ICCs”).

In some embodiments the cells are transgenic cells, such as cells that have been transformed to express a protein of interest, such as one or more colony-stimulating factors, erythropoietin, growth hormone, insulin, interferon, HGF, or plasminogen activators. In some embodiments cells that have been transformed express a small molecule of interest, such as one or more small molecule hormones of interest, such as one or more steroid hormones. In some embodiments the molecule is a native human molecule. In some embodiments the molecule is a synthetic molecule, such as a molecule having an effect that is greater than, less than, or different from that of a related native molecule, or a synthetic molecule that is unrelated to a native human molecule.

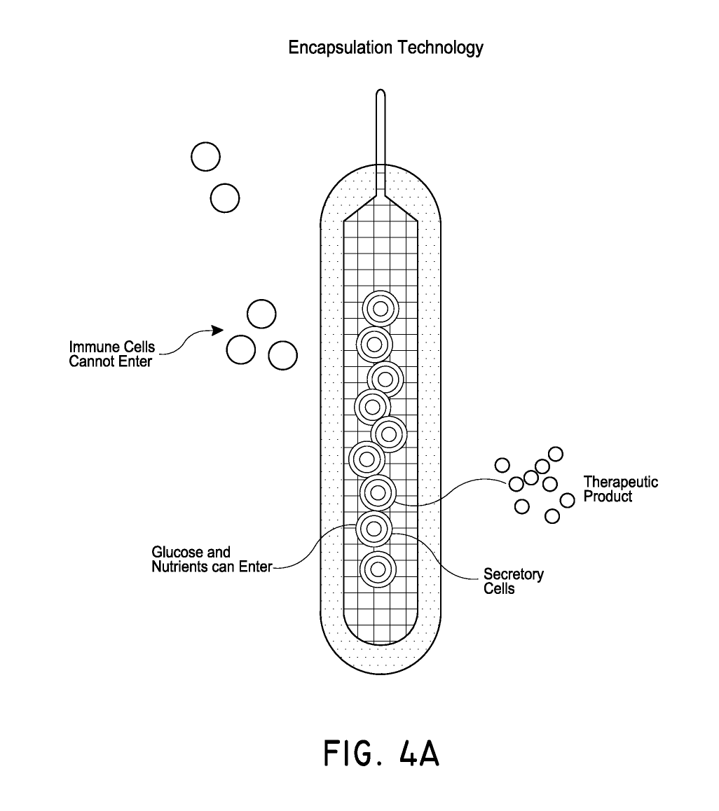

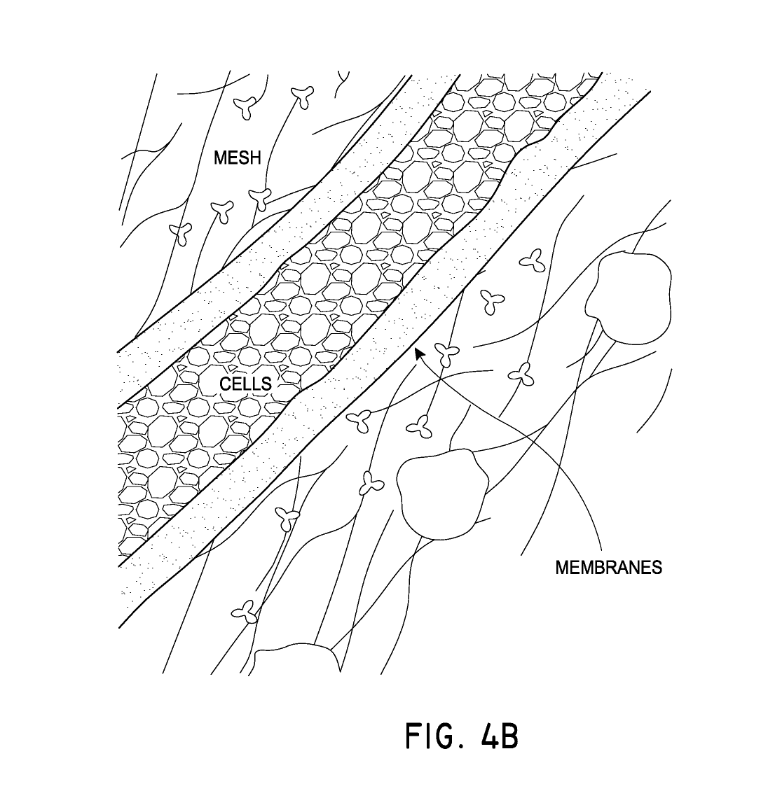

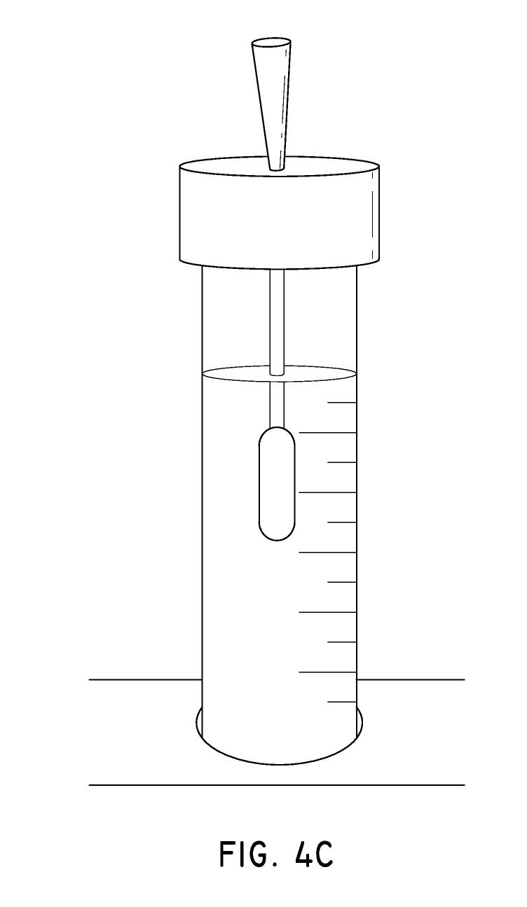

Some embodiments involve transplanting embryonic stem cell derived pancreatic islet progenitors. Because of concerns over the tumorigenicity potential of any pluripotent cells contaminating pancreatic cell preparations, a durable macro-encapsulation device (Loudovaris et al. (1999) J Mol Med, 77) offers a number of advantages. The general principles of the immunoisolating macro-encapsulation technology are shown in FIG. 4A and reviewed in (O'Sullivan et al. (2011) Endocr Rev, 32:827-844). Remarkably, in studies with encapsulated insulin producing cells, diabetes was reversed even when devices were placed subcutaneously (Lee et al. (2009) Transplantation, 87:983-91; Tarantal et al. (2009) Transplantation, 88).

In some embodiments, the device retains its integrity when inserted into the body. In some embodiments the device retains its integrity when inserted into the body such that encapsulated cells will not escape into the host. In some embodiments the device is Teflon-like and therefore virtually unbreakable.

In some embodiments, the device is retrievable. This is a desirable feature in the event of an adverse reaction.

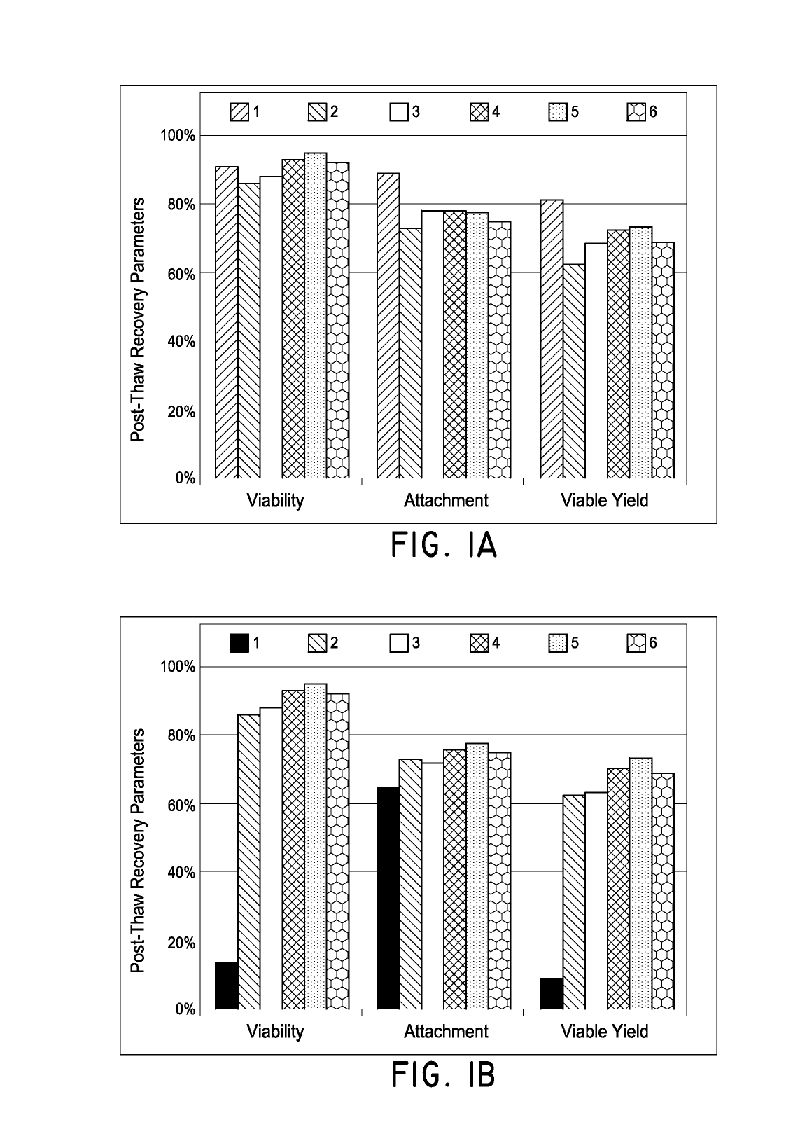

The sensitivity of dissociated human Ins-G cells to DMSO without cryopreservation (CP) was investigated. The results shown in FIG. 1A indicated that exposure of monolayers cultured cells to 10% DMSO was well tolerated before for up to 30 minutes, the longest time tested. Temperatures ranging from 0° C. to 37° C. were investigated. Regardless, cell viability was practically indistinguishable from control cells not exposed to DMSO (FIG. 1A, column #1).

The effects of preincubation with DMSO on freeze/thaw viability were investigated. Freezing dissociated cells pre-exposed to Freeze Media containing 10% DMSO (FIG. 1B) also had no effect on cryosurvival, which per se was exceptionally good (up to 77-79% to control). In order to verify requirement for cryoprotective agents (CPA) in freezing, a sample was frozen in culture media (no DMSO). As expected, survival was very poor (11% in comparison to the positive control). Interestingly, the cells re-attached to the plate surface after thawing, but died in the monolayer. Without being bound to theory, a possible explanation is that cell mortality resulted from slow (suboptimal) freezing injury, when cells are frozen at the rate substantially slower than the equilibrium “Mazur's rate” when the cells are just in equilibrium with freezing outer milieu. The suboptimal rates in the inverted U curve usually contributed to the effects of highly concentrated solutes rather than immediate cell destruction and fast necrosis associated with the supra-optimal (fast freezing, according to the 2-factor hypothesis developed by Mazur (Mazur et al. (1972) Experimental Cell Res., 71:345-349; Mazur et al. (2008) Biol Reprod, 78:2-12). The cell membrane rupture and other colligative osmotic damage due to excessive shrinkage and swelling usually manifest quickly as the loss of selective permeability properties so the cells become permeable to large particle dyes such Trypan Blue, soon after the treatment. The osmotic damage usually decreases with the temperature and step-wise addition and dilution (which, in fact, exposes the cells to CPA longer than an abrupt one-step addition or dilution). In contrast, a long exposure to a CPA or/and to very concentrated solutions of the cryoprotectant can manifest later in the form of, e.g., death due to specific chemical toxicity, particularly on membrane and epigenetic level as it may affect protein folding and membrane lipid bilayers, as it is shown for some types of cells (see, for example (Katkov II et al. (2006) Cryobiology, 53:194-205) for references. In contrast to osmotic damage, chemical toxicity usually exacerbates at longer exposure at higher temperature, and the prevalence of one mode vs. another can be easily differentiate by treating cells with higher temperatures and/or for longer exposure to the CPA (Katkov II (2011) Cryobiology, 62:242-4; Katkov II et al. (1998) Cryobiology, 37:325-38; Katkov II et al. (2007) Cryo Letters, 28:409-27).

Another indication that the freezing rate was suboptimal was by comparison with the perfect cryosurvival of cells frozen with DMSO vs. very poor survival without the CPA, and it is known that permeable CPAs mainly protect from the slow injury working as osmotic buffers and preventing the excessive shrinkage and dehydration of the cells during freezing (Katkov II et al. (2012) in Current Frontiers in Cryobiology http://www.intechopen.com/books/current-frontiers-in-cryobiology, ed. I.I. Katkov (InTech, pp. 3-40); Lovelock J E (1953) Biochim Biophys Acta, 11:28-36; Mazur P (1970) Science, 168:939-49). As a general conclusion, cells cultured as monolayer and dissociated before the cryo procedure perfectly tolerated both long (30 min) and warm (+37° C.) exposure to DMSO before CP as well as CP per se, which excludes possible chemical toxicity of DMSO to this type of cells.

To more closely mimic the morphological structure of pancreatic islets, cells cultured as 3-dimensional ICCs were investigated. The first technical question was how to harvest clusters, whether by gentle (200 g×5 min) centrifugation or by settling by gravitational force (1 g). Here prefreeze exposure of the clusters to 10% DMSO at room temperature and +37° C. before freezing was investigated.

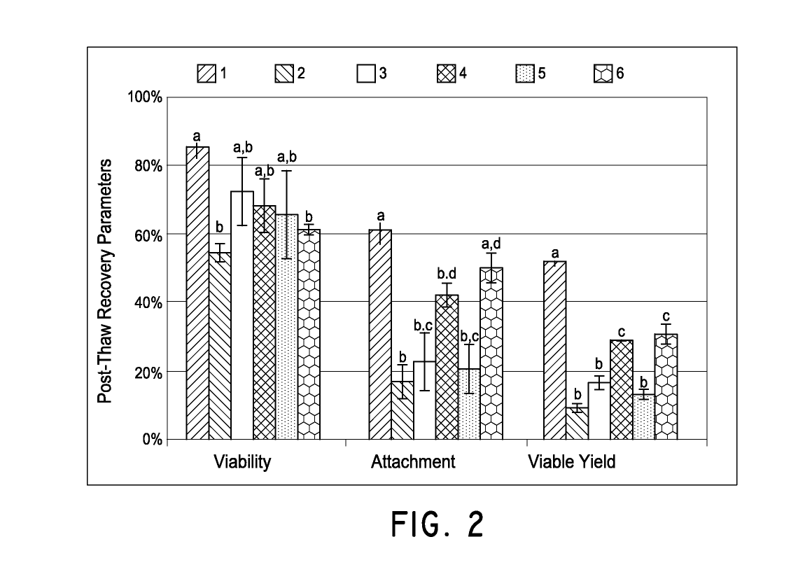

Two negative controls were employed. First (sample 1 on FIG. 2) were non-frozen clusters but the cells were exposed to 10% of DMSO for 20 min at +37° C. and centrifuged (200 g×5 min) The second control (Column #2) were cells that were not exposed top DMSO and frozen w/o it, with the same other procedures that in the first control. In contrast to the cells grown in monolayers and frozen in dissociated state) clusterization of the cells significantly impeded their viable yield, ranging between 9% for frozen clusters w/o DMSO 31% (clusters exposed for at +37° C. for 20 min and centrifuged 200 g×5 min). Prior reports indicated that centrifugation could damage sensitive cells (Katkov II et al. (1998) J Androl, 19:232-41). Interestingly however, viability of attached clusters was essentially the same, but attachment ability was substantially lower in 1 g samples. Apparently, some clusters were lost because they had not settled to the bottom of the tube either due to sub-optimal time or length of column 15-mL tube (Katkov II et al. (1999) Cell Biochem Biophys, 31:231-45). Cell clusters, it was observed, are more sensitive than dissociated cells from monolayer cultures even before and freezing (viability after thawing for the re-attached cells was in range of 61-72%, which was substantially lower than in monolayers (Experiment 2, FIG. 2). Thus, natural sedimentation was used with an increased time and decreased column length by using 50-mL conical tubes instead for the next experiments.

As the cells in the device are exposed to DMSO for a relatively long time both during loading and after thawing (elution of DMSO from a large macro-device is significantly slower than from small clusters or especially single cells) an important question is post-thaw toxicity, e.g. exposure to DMSO after thawing. While the level of DMSO will gradually decrease during elution, the extreme point was tested and the same high level of DMSO was maintained for the longevity of the entire exposure.

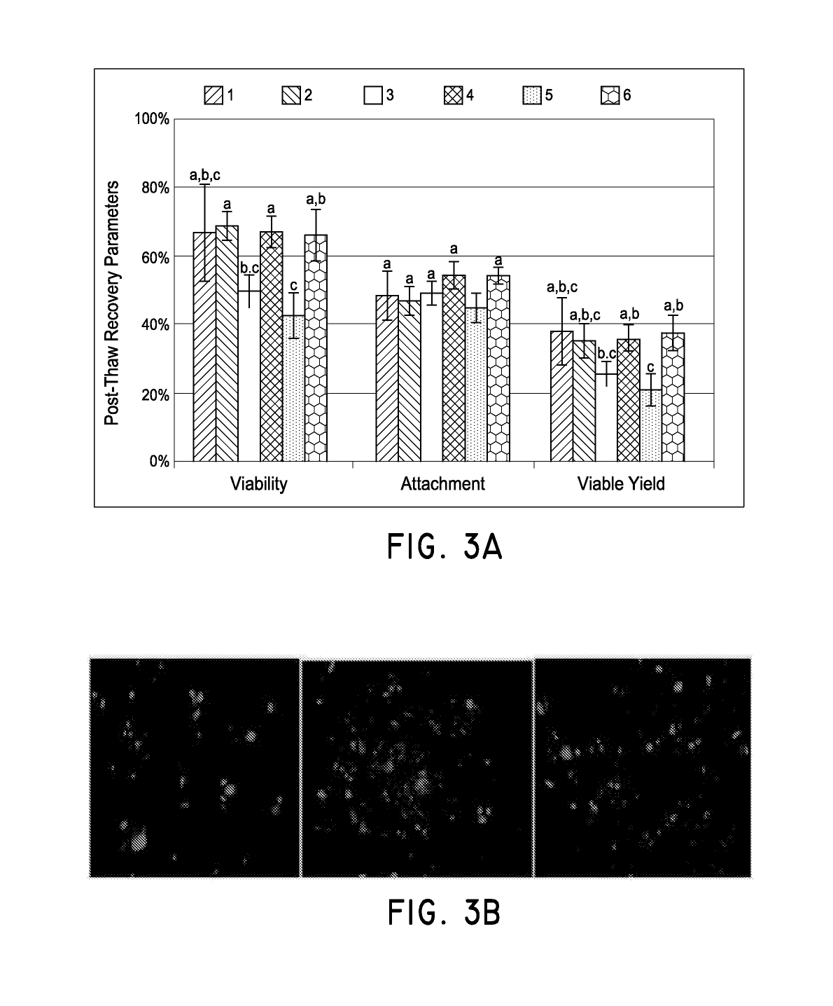

Increase of temperature of elution will shorten the time of elution but DMSO might cause increased toxicity at increased temperature. Driven by this dilemma, extreme treatments of post-thawed clusters were checked starting with immediate washing of DMSO, maintaining them for 20 min on 4° C. before washing, at 25° C. for 10 and 20 min and at +37° C. at 10 and 20 min (Experiment 3, FIG. 3A). Surprisingly, the time and temperature of exposure did not alter Trypan Blue exclusion the attached cells, (% of attached cells, and the viable yield). The cells that were exposed to +37° C. for 20 minutes tolerated equally well or even slightly, but not significantly, better than any other treatment including the clusters that were washed immediately after thawing (viable yield in range 35-38% across the board). There was one exception however, the cells incubated at room temperature for 20 minutes performed slightly poorer than the other treatments. They exhibited lower attachment ability and viability of the reattached cells, which culminated in much lower (21%) viable yield. This phenomenon was observed over a large number of experiments (N=6), but its basis is unclear. However, due to high variability among experiments for all treatments makes the difference not statistically significant, mostly because of one particular experiment, when that treatment showed particularly low results (which can be accidental). Further experiments would be needed to determine with certainty that RT is actually more detrimental than +37° C. or ice. The surviving cells retain insulin expressing capacity for all treatments (FIG. 3B).

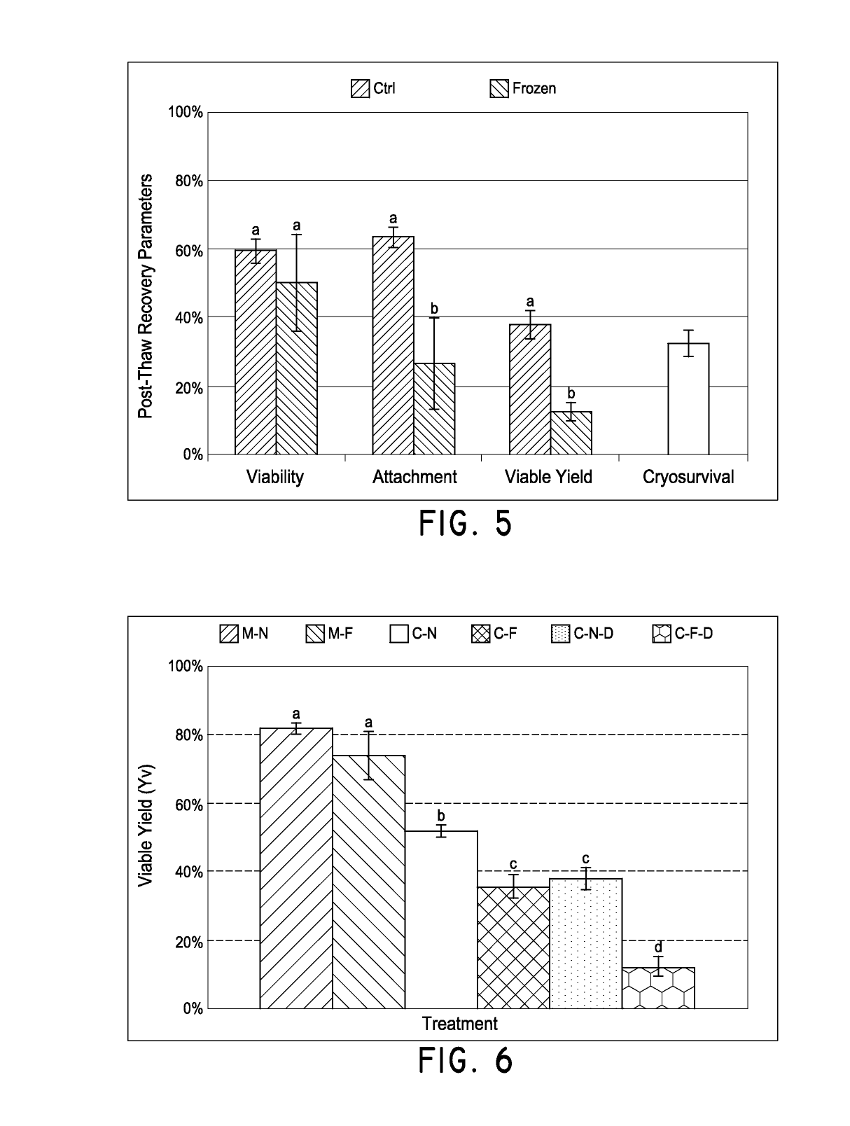

Observing that the clusters can be exposed to DMSO both before freezing and after thawing when the cells were frozen in cryovials, cells were then frozen in the TheraCyte™ device (FIGS. 4A-C). Cells were exposed to DMSO during loading into the device. The port was then trimmed so that the device could fit a 1.5 mL cryovial which also contained 10-% DMSO freezing medium and froze them. After the vial was thawed, DMSO was eluted by placing the device in gently swirling medium solution (using magnetic stir bar). Without being limited by theory, this treatment may be sufficient to wash out the majority of DMSO before transplantation. Moreover this procedure mimics slow elution if the device is transplanted immediately after thawing, as a low concentration of DMSO is presumed to be non-toxic even for small animals. Survival however was significantly reduced when compared with unencapsulated clusters. Barely 1/10 of loaded cells survived the whole procedure (including cutting device in pieces and placing into the dish, washing that out in 48 hrs, etc. (Experiment 4, FIG. 5). There can be several explanations for the cell loss, such as following.

1. Some viable cells may have been lost during the loading and removal procedures. The device per se was not visibly damaged by freezing, as we did not observe cells in the wash solution, when the device was exposed to laminar flow for 30 min. Had the device been severely compromised, cells would have escaped into the elusion media. It was not yet investigated as to whether the device maintained its complete immunoisolating properties, but no visible cracks or change of the robust mesh structure were observed. Yet, loading and unloading cells per se can lead to certain loss of cells. That is however also true for the non-frozen device. Consequently both were compared, keeping the non-frozen device as the baseline for the amount of loaded cells. In any case, it will need more vigorous experiments.

2. The mesh, in contrast to alginate, is robust, which is a substantial advantage. It might, however not provide cell-to-matrix interaction that can be present in microcapsules. That interaction might help to compensate for detachment induced apoptosis anoikis, as it presumably happens in case of human ESCs (Krawetz et al. (2009) Bioessays, 31:336-43; Wagh et al. (2011) Stem Cell Rev, 7:506-517), or through other pathways related to death of non matrix bound cells (Ichikawa et al. (2012) Cryobiology, 64:12-22; Ichikawa et al. (2011) Cryo Letters, 32:516-24).

3. The protocol of freezing was straightforward for CP of single cells and not adjusted to the size of the clusters, which can be a crucial factor in developing initial rate of freezing (Schneider et al. (2005) Diabetes, 54:687-691). Thus, using an elaborate multi-step protocol as is done by Schneider and Klien is contemplated in some embodiments (Schneider et al. (2011) Eur J Med Res, 16:396-400; Schneider et al. (2011) Regul Pept, 166:135-138).

4. Clusterization per se decreased the cell survival even for unfrozen cells. Loading and preloading further decreased viability even without freezing. Altogether, as each step was not individually optimized, the decrease in Viable Yield had a cumulative effect. This is summarized (FIG. 6), where it is clearly seen that clusterization “-C” label) and loading clusters in device (“-D” label) even for non-frozen cells (cf. columns with “-N” labels) was in comparison with very robust cells grown in monolayers (“-M” label). Cryopreservation (“-F” labels) further amplified that effect. It indicates that all steps of the technology would need further optimization. However, these cells are normally grown in monolayers, so clusterization may itself be harmful. In experience with ICCs derived from stem cells, macro-encapsulation into TheraCyte™ devices did not appear to be harmful to them (unpublished results). This may be because they are more tightly connected to each other. Therefore, it is contemplated that stem cell derived ICCs may survive freezing even better than human Ins-G cells employed here.

Freezing islet like clusters of insulin expressing Ins-G cells, was accomplished and is disclosed herein, either unencapsulated or encapsulated in a clinically tested immune macro-encapsulation device. A heuristic strategy has been explored (small number of experiments with large number of experimental variations) as the goal was a proof of principle, and live and fully functional cells have been obtained following encapsulation, freezing, removal from the device, and replating. Further investigations are necessary to elucidate the mechanisms for the degree of cell loss described in this report so that protocols can be optimized. Cryopreserved encapsulated insulin expressing cells or their precursors may prove a valuable method for dispensing the next generation of cell based therapy, such as cell therapy for diabetes. These findings may also be of more general value as this is the first report in cryopreservation of cells macro-encapsulated into a durable therapeutic device, and the lessons we have learned for development of for general cryopreservation strategies can be used for numerous regenerative medicine based therapies. There is much room for improvement and investigation of the characteristics of both the target cells and the immunoisolating devices (permeability to water and DMSO, for example) but we are quite optimistic and think that the cells can and should be cryopreserved in macro-encapsulation devices.

Alternative cell freezing protocols are contemplated herein. For example, depending on the cell type, one may vary DMSO concentration, freezing time, and freezing temperature, for example, to obtain best results for a given culture. Cells may be frozen in medium comprising constituents such as cryoprotective agents other than DMSO, such as glycerol, propanediol, ethylene glycol, sugars, or other macromolecules. The concentration of the cyroprotectant, and of other constituents of the composition, may be varied to optimize cell survival or other parameters. Cells may be frozen in, for example, phosphate buffered saline, trypsin/EDTA solution, a zwitterionic buffer solution such as HEPES, TES or TRIS, alone or in combination. A number of temperature regimens may be employed, such as rapid freezing, flash freezing, or slow freezing. Similarly, thawing may be rapid or slow, and may comprise one or more intermediate freezing or thawing steps. Additionally or in combination, exposure to the cryoprotectant may be gradual or immediate, and upon thawing the cryoprotectant may be removed either stepwise or all at once from the revived cells. Other cell freezing and thawing protocols are contemplated, and may be customized to match the cell population in question. Use of a number of cell freezing protocols and compositions is consistent with the disclosure herein.

Referring again to FIG. 4A, one sees an operating principle of the present disclosure. Secretory cells are contained within a device that is permeable to glucose, nutrients and at least one therapeutic product, but impermeable to the secretory cells contained therein or the immune cells excluded from the interior of the device.

While embodiments disclosed herein have been described in some detail for purposes of clarity and understanding, one skilled in the art will appreciate that various changes in form and detail can be made without departing from the true scope of the disclosure.

### EXAMPLES

### Example 1

### Pre-Freeze Exposure of Dissociated Human Ins-G Cells to Cryoprotective Agent (:10% DMSO)

In prior studies with a macro-encapsulation device, we loaded cells into devices immediately prior to transplantation. Thus, until now we have not exposed encapsulated cells to CPA, yet the ability to freeze preloaded devices would ensure reproducibility between experiments and among users. The process of loading cells into the device requires approximately 20 min; beginning when cells are drawn up into a Hamilton syringe and slowly dispensed into the device, and ending when the syringe has been removed from the device port and the port has been sealed. We hypothesized therefore, that cells must withstand pre-freeze exposure to CPA for 20 minutes in order to be successfully cryopreserved in devices. Thus, we began to investigate CPA toxicity on the islet derived cell line Ins-G (Itkin-Ansari et al. (2005) Developmental Dynamics, 233:946-953; Kiselyuk et al. (2010) J Biomol Screen, 15:663-670).

Cells cultured as monolayers were dissociated with Accutase, pelleted, and resuspended with either normal culture media (CM) as a positive control for cell viability or with the Freeze Medium (FM) containing 10% DMSO. Cells were either plated immediately in CM or incubated in FM for up to 30 minutes. Following incubation time, cells were allowed to reattach to tissue culture plates for 24 hours and then assessed for attachment and viability. As expected, control cells cultured in CM exhibited high attachment efficiency (89%) and high viability (91%) (FIG. 1A, column marked as “1”). Cells exposed to FM for 1 minute at 4° C. exhibited slightly slower attachment ability (73-78%) yet survival of attached cells was similar to controls. Interestingly, neither attachment nor viability was significantly reduced by exposure to 10% DMSO under any conditions tested both in regards to the length of exposure to DMSO (10 or 30 min) or temperature of the FM (4° C. or 37° C.). Cells in all conditions exhibited survival in a range from it 86-93% which did not statistically differ from the control cells (91%). As a result, the Viable Yield (the product of Attachment and Viability) was also similar among the 4 incubation conditions 3-6 (63-73%). Thus, exposure to DMSO without freezing produced a minimal effect on survival of dissociated cells monolayer cultures.

The viability of cryopreserved dissociated cells was not affected by pre-freeze exposure to 10%-DMSO regardless of time (10 or 30 min) or temperature of exposure (+4° C. or +37° C.). The viability of re-attached cells after freezing was 86-92% and the Attachment efficiency was in range 73-77%, resulting in a Viable Yield 63-73%.

### Example 2

### Effect of Exposure of Islet-Like Cell Clusters (ICCs) to 10% DMSO and Centrifugation Prior to Freezing

Results of Experiment 1 on dissociated cells suggested that exposure to CPA for up to 30 minutes was not detrimental to cells. However, human islets exist as 3-dimensional clusters of 500 to 1000 cells and the kinetics of CPA diffusion through cell clusters will necessarily differ from single cells. Because we ultimately plan to encapsulate islets or like-cell clusters (ICCs), we chose to mimic the morphology of islets in Experiment 2. Human Ins-G cells cultured in non-adherent plates spontaneously form islet like cell clusters (ICCs) (Itkin-Ansari et al. (2003) Ann N Y Acad Sci, 1005:138-147; Itkin-Ansari et al. (2005) Developmental Dynamics, 233:946-953). Here ICCs were exposed to 10% DMSO for 20 minutes at 25° C. or 37° C. Clusters were then pelleted either by passive settling via gravity or by centrifugation prior to freezing. Centrifugation prior to freezing enhanced attachment of cells post-thaw (probably due to complete recovery of small clusters). There was no significant difference between pre-freeze exposure to CPA at 25° C. versus 37° C.

### Example 3

### Survival and Function of ICCs Following Post-Thaw Exposure to 10% DMSO

Ultimately, the ability to freeze cell clusters inside an encapsulation device may require that the cells survive the time required to remove CPA upon thawing. Normally, when (unencapsulated) cells are thawed, CPA is removed quickly, either by centrifugation or by extensive dilution into culture media. However, for a cell-filled device, CPA removal will be accomplished via diffusion of CPA out of device into a large volume of wash media, until equilibration is reached. We estimated that approximately 20 minutes will be required to reach equilibrium, during which time cells are exposed to CPA. We therefore first examined the effects of post-thaw exposure to CPA in unencapsulated ICCs. Because equilibrium is also temperature dependent, we exposed thawed ICCs to CPA at 25° C. or 37° C. As we can see from FIG. 3A, sample 1, a control which was not exposed to CPA or frozen, reattachment of ICCs to tissue culture plates is 48%, lower than reattachment of dissociated cells (FIG. 1, sample 1). While there was a trend toward lower viable yield in cells exposed to DMSO for 20 minutes at 25° C., statistical significance was not reached. Thus, post-thaw exposure to DMSO was well tolerated.

In addition to survival, it is important that ICCs retain insulin gene expression during these manipulations. Human Ins-G cells express endogenous insulin message in response to tamoxifen (which activates the transcription factor E47 by promoting translocation from the cytoplasm to the nucleus). The cells were also engineered to express GFP from an insulin transgene. Therefore, GFP expression in response to tamoxifen is a surrogate for insulin expression. We examined GFP expression in control ICCs (not frozen, not exposed to DMSO) as well as in cells frozen and exposed to DMSO for 20 minutes at 37° C. (FIG. 3B). There was no significant difference in GFP expression between cultures. Therefore, freezing ICCs and exposing them to DMSO post-thaw does not significantly diminish viable yield or function.

### Example 4

### Freezing ICCs in a Immunoisolating Device

We next addressed the effects of freezing ICCs inside the encapsulation device. A schematic view of the encapsulation device is shown in FIG. 4A. An image of an actual device suspended in a 50 mL conical containing media is shown in FIG. 4B. As a control for freezing, ICCs resuspended in FM were loaded into devices which were sealed and then immediately opened and cells removed. Cell attachment following release from device averaged 64% and of the attached cells, 59% were viable. Thus, the Viable Yield was 38%. The data suggest that the process of loading ICCs into the device was no more damaging than exposure DMSO alone (Compare FIG. 4C with FIG. 2). In contrast, freezing cells in devices reduced Viable Yield. Devices which had been frozen were thawed and allowed to reach equilibrium in wash media for 20 min at 25° C. with laminar flow. Devices did not appear to be damaged by freezing, as we were unable to detect cells in the wash media. Had the device been severely damaged, cells would have escaped into the wash media. When cells were removed from the device post thaw however, cell attachment to tissue culture plates averaged 50% and of those, 26% were viable, therefore the Viable Yield was 12%. In summary, the Cryorecovery (Viable Yield of Frozen/Control) of cells frozen in the device was 32%, suggesting that with protocol optimization, freezing preloaded devices will be an attainable goal.

### Example 5

### Effect of Exposure of hESCs to Cryoprotective Agent

hESCs are cultured as monolayers and are dissociated with Accutase, pelleted, and resuspended with either normal culture media (CM) as a positive control for cell viability or with the Freeze Medium (FM) containing 10% DMSO. Cells are either plated immediately in CM or incubated in FM for up to 30 minutes. Following incubation time, cells are allowed to reattach to tissue culture plates for 24 hours and then assessed for attachment and viability. DMSO concentration, freezing time, and freezing temperature, for example, may be varied to obtain best results for a given culture. It is expected that control cells cultured in CM exhibit high attachment efficiency and high viability. Cells exposed to FM for 1 minute at 4° C. may exhibit slightly slower attachment ability yet survival of attached cells may be similar to controls. Neither attachment nor viability is significantly reduced by exposure to 10% DMSO under any conditions tested both in regards to the length of exposure to DMSO (10 or 30 min) or temperature of the FM (4° C. or 37° C.). Cells in all conditions exhibit survival in a high range such as 80% or higher which may not statistically differ from the control cells. As a result, the Viable Yield (the product of Attachment and Viability) is also similar among the 4 incubation conditions. Thus, exposure to DMSO without freezing produces a minimal effect on survival of dissociated cells monolayer cultures.

### Example 6

### Freezing hESCs in a Immunoisolating Device

We address the effects of freezing hESCs inside the encapsulation device. A schematic view of the encapsulation device is shown in FIG. 4A. An image of an actual device suspended in a 50 mL conical containing media is shown in FIG. 4B. As a control for freezing, hESCs resuspended in FM are loaded into devices which are sealed and then immediately opened and cells removed. Cell attachment following release from device averages over half of the cell population, and of the attached cells, over half are viable. Thus, the Viable Yield is near 50%. The data suggest that the process of loading hESCs into the device is no more damaging than exposure DMSO alone.

In contrast, freezing cells in devices reduces Viable Yield. Devices which had been frozen are thawed and allowed to reach equilibrium in wash media for 20 min at 25° C. with laminar flow. Devices do not appear to be damaged by freezing, as we are unable to detect cells in the wash media. When cells are removed from the device post thaw however, cell attachment to tissue culture plates averages about half and of those, about ¼ are viable, therefore the Viable Yield is about ⅛. In summary, the Cryorecovery (Viable Yield of Frozen/Control) of cells frozen in the device is about ⅓, suggesting that freezing preloaded devices is a viable course of action.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present disclosure. These embodiments are susceptible to modifications in the methods and materials, as well as alterations in the fabrication methods and equipment. Such modifications will become apparent to those skilled in the art from a consideration of this disclosure or practice of the embodiments disclosed herein. Consequently, it is not intended that this disclosure be limited to the specific embodiments disclosed herein, but that it cover all modifications and alternatives coming within the true scope and spirit of the disclosure.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1. A macro encapsulation device comprising frozen cells capable of secreting a therapeutic molecule.

2. The device of claim 1, wherein at least about 30% of the cells are viable after thawing.

3. The device of claim 1, wherein said cells are frozen islet like cell clusters.

4. The device of claim 1, wherein at least about 30% of the cells in said islet like cell clusters are viable after thawing.

5. The device of claims 1 wherein the therapeutic molecule is a protein or a hormone.

6. (canceled)

7. A method of cryopreserving viable cells which secrete a therapeutic molecule, comprising contacting said cells with DMSO and freezing said cells.

8. The method of claim 7 further comprising thawing said cells which secrete a therapeutic molecule such that said thawed cells which secrete a therapeutic molecule comprise viable cells.

9. The method of claim 7 wherein said DMSO is a constituent of a composition that comprises 10% DMSO.

10. The method of claim 7 further comprising transporting said frozen cells which secrete a therapeutic molecule to a clinical setting.

11. The method of claim 7 wherein said cells which secrete a therapeutic molecule are preloaded into a cryopreservation device before freezing.

12. (canceled)

13. The method of claim 7 wherein said cells which secrete a therapeutic molecule are frozen before loading into a cryopreservation device.

14. The method of claim 12 wherein said freezing is partial or total.

15. (canceled)

16. The method of claim 7 further comprising transplanting said cells which secrete a therapeutic molecule into a patient.

17. (canceled)

18. The method of claim 7 wherein said cells which secrete a therapeutic molecule are maintained in said DMSO for up to 20 minutes after thawing.

19. The method of any of claim 7 wherein said cells which secrete a therapeutic molecule are maintained at a temperature of up to 37° C. for up to 20 minutes.

20. The method of claim 7, wherein said cells which secrete a therapeutic molecule are cells in islet like cell clusters.

21. A composition comprising DMSO and islet like cell clusters wherein said composition is frozen and wherein said islet like cell clusters are viable.

22. The composition of claim 21 further comprising a cryopreservation device.

23. The composition of claim 22 wherein said cryopreservation device is impermeable to said islet like cell clusters.

24. The composition of claim 22 wherein said cryopreservation device is permeable to at least one substance secreted by said islet like cell clusters.

25. The composition of claim 24 wherein said substance is insulin.

26. The composition of claim 22 wherein said cryopreservation device is configured to maintain its integrity upon subcutaneous introduction into a mammal.

27-50. (canceled)
